(19) United States
(12) Patent Application Publication
(10) Pub. No.: US 2005/0027236 A1
(43) Pub. Date: Feb. 3, 2005

(54) Aspiration catheter having a variable over-the-wire length and methods of use

(75) Inventor: Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic AVE, Inc.

(21) Appl. No.: 10/629,824

(22) Filed: Jul. 30, 2003

(51) Int. Cl. A61M 3/02

(52) U.S. Cl. 604/40

(57) ABSTRACT

An aspiration catheter includes an aspiration shaft and a telescoping outer sheath which can be passed over a guidewire. The sheath includes “nesting” proximal and distal tubes. The distal tube slides within a proximal tube lumen, and the aspiration shaft slides within a distal tube lumen. The catheter can be placed in an expanded position by pushing the aspiration shaft, thereby causing the distal tube to be moved distally. The catheter can be retracted by pulling the aspiration shaft proximally so that a distal segment of the aspiration shaft is drawn into the distal tube lumen and the distal tube is drawn into the proximal tube lumen. Alternatively, a middle tube is slidingly disposed over the aspiration shaft between the proximal and distal tubes. In this embodiment, the middle tube nests within the proximal tube lumen and the distal tube nests within a middle tube lumen.

### BACKGROUND OF THE INVENTION

1. Field of the Invention

This invention relates to catheters for use within a body of a patient, and more particularly to aspiration catheters for removing debris from a body lumen.

2. Background of the Invention

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent that is placed in the body on a permanent or semi-permanent basis to support weakened or diseased vascular walls to avoid catastrophic rupture thereof.

Often, more than one interventional catheter is used during a procedure, such as to change the size of the balloon being used or to introduce additional devices into the system to aid with the procedure. In such situations, the catheters are generally inserted into the patient's cardiovascular system with the assistance of a guidewire. For example, a guidewire is introduced into the patient, steered through the tortuous pathways of the cardiovascular system, and positioned at a predetermined location. Various catheters having a guidewire lumen adapted to receive the guidewire may then be introduced into and removed from the patient along the guidewire, thereby decreasing the time needed to complete a procedure.

The treatment or removal of stenosis may introduce thrombi and/or emboli into the bloodstream. These particles can actually worsen a patient's condition by blocking the body lumen in the vicinity of the treatment area, or the particles can migrate to other parts of the body and create blockages in those areas. If the body lumen becomes occluded, the patient may suffer such effects as myocardial infarction or stroke.

Many techniques exist for preventing the introduction of thrombotic or embolic particles into the bloodstream during such a procedure. Common among these techniques is to introduce an occluding device or a filter downstream of the treatment area to capture these embolic or thrombotic particles. The particles may then be removed from the vessel with the withdrawal of the occluding or filtering device, or the particles may be removed prior to the withdrawal of these devices using an aspiration catheter.

An aspiration catheter includes a tubular body having an aspiration lumen and is typically of the “over-the-wire” variety. Thus the aspiration catheter may be designed such that a guidewire is contained within the aspiration lumen as the catheter is advanced thereover, or the aspiration catheter may include a guidewire shaft extending along substantially the entire length of the aspiration catheter such that the guidewire is disposed therein as the catheter is advanced through a body lumen. Each of this type of over-the-wire aspiration catheter is shown in U.S. Pat. No. 6,152,909 to Bagaoisan et al. which is incorporated herein in its entirety by reference thereto.

While these over-the-wire catheters are advantageous in many ways, deploying and exchanging the aspiration catheter can be difficult. In order to maintain a guidewire in position while withdrawing an indwelling aspiration catheter, the clinician must grip the proximal end of the guidewire to prevent it from becoming dislodged during removal of the aspiration catheter. However, the aspiration catheter, which is typically on the order of 135 centimeters long, is generally longer than the exposed portion of the guidewire. Therefore, to be able to maintain the guidewire in place, the guidewire must be sufficiently long so that the clinician may be able to maintain a grip on an exposed portion of the guidewire. For aspiration catheters on the order of 135 centimeters in length, therefore, a guidewire of 300 centimeters in length is necessary. Manipulating an aspiration catheter along such a long guidewire typically requires more than one operator, thereby increasing the time and complexity of the procedure.

Many techniques have been used to overcome this problem. For example, a guidewire of a shorter length is used during the procedure, but during the exchange process, such as when an indwelling therapeutic catheter is exchanged for an aspiration catheter or when an indwelling aspiration catheter is exchanged for a therapeutic catheter, a longer exchange guidewire is substituted for the original guidewire. Also, as is disclosed in U.S. Pat. No. 4,917,103 to Gambale et al., incorporated herein in its entirety by reference thereto, the length of the original guidewire may be extended using a guidewire extension apparatus. However, neither of these techniques eliminates the need for more than one operator to complete the procedure.

Aspiration catheters may also be of the single operator or “rapid-exchange” type. A rapid-exchange aspiration catheter typically includes a tubular body with an aspiration lumen extending the entire length thereof and a guidewire shaft having a guidewire lumen of minimal length positioned along a distal portion of the catheter, although some of these catheters are not advanced over guidewires at all. As such, the guidewire is located outside of the aspiration catheter except for a short segment which runs within the guidewire lumen. Therefore, a clinician is able to control both ends of the guidewire while the aspiration catheter is loaded onto the guidewire. The aspiration catheter is then advanced through the patient with only a distal portion of the catheter riding on the guidewire. U.S. Pat. No. 6,152,909 to Bagaoisan et al. also describes this type of aspiration catheter.

While convenient for rapid and simple exchange, rapid-exchange type catheters typically lack the desired stiffness and pushability for readily advancing the catheter through the tortuous vascular system. Furthermore, use of these catheters increases the likelihood of guidewire entanglement and may lead to damage of the vascular walls due to the tension load applied to the guidewire. Although a single clinician may be able to deploy such an aspiration catheter, the long proximal end of the catheter is still relatively difficult to manipulate, thereby increasing the complexity and duration of the deployment of the aspiration catheter.

Various techniques have also focused on adjusting the length of a catheter so that the length thereof can be varied, such as to reduce the length of the catheter during a catheter exchange. U.S. Pat. No. 5,591,194 to Berthiaume, incorporated herein in its entirety by reference thereto, describes an over-the-wire balloon catheter with an adjustable length. The balloon catheter includes several telescoping portions slidably mounted on an inflation shaft which is fixedly attached to the distal balloon. The telescoping portions may be retracted by drawing the inflation shaft backwards, thereby reducing the effective over-the-wire length of the telescoping balloon catheter. As such, the balloon catheter may be withdrawn from the patient without using an unnecessarily long guidewire. However, this patent does not disclose telescoping catheter technology for use with an aspiration catheter.

### SUMMARY OF THE INVENTION

Accordingly, disclosed herein is an aspiration catheter with an aspiration shaft and a telescoping outer sheath. The sheath includes “nesting” proximal and distal tubes. Each tube has a single lumen and is slidably disposed over the aspiration shaft. The distal tube is sized to slide proximally and distally within the proximal tube lumen, and the aspiration shaft is sized to slide proximally and distally within a distal tube lumen. Further, the distal tube cannot be completely extracted from the proximal tube lumen, and the aspiration shaft cannot be completely extracted from the distal tube lumen. As such, the catheter can be placed in an expanded position either by pulling the distal end of the aspiration shaft or by pushing the aspiration shaft, thereby causing the distal tube to be moved distally. The catheter can be retracted to a rapid-exchange length by pulling the aspiration shaft proximally so that the distal tube is drawn into the proximal tube lumen, thereby shortening the effective over-the-wire length of the catheter.

### BRIEF DESCRIPTION OF THE DRAWINGS/FIGURES

The accompanying drawings, which are incorporated herein and form a part of the specification, illustrate the present invention and, together with the description, further serve to explain the principles of the invention and to enable a person skilled in the pertinent art to make and use the invention.

FIG. 1 is a longitudinal cross-sectional view of an aspiration catheter system according to an embodiment of the present invention in a fully extended position.

FIG. 2 is an enlarged view of a joint area of a proximal tube and a distal tube of the embodiment of FIG. 1.

FIG. 3 is a longitudinal cross-sectional view of the embodiment of FIG. 1 in a rapid-exchange position.

FIG. 4 is a longitudinal cross-sectional view of an alternate embodiment of an aspiration catheter system according to the present invention in a fully extended position.

FIG. 5 is a longitudinal cross-sectional view of the embodiment of FIG. 4 in a rapid-exchange position.

FIG. 6 is a longitudinal cross-sectional view of an alternate embodiment of an aspiration catheter system according to the present invention in a fully extended position.

FIG. 7 is a longitudinal cross-sectional view of the embodiment of FIG. 6 in a rapid-exchange position.

### DETAILED DESCRIPTION OF THE INVENTION

Specific embodiments of the present invention are now described with reference to the figures, where like reference numbers indicate identical or functionally similar elements.

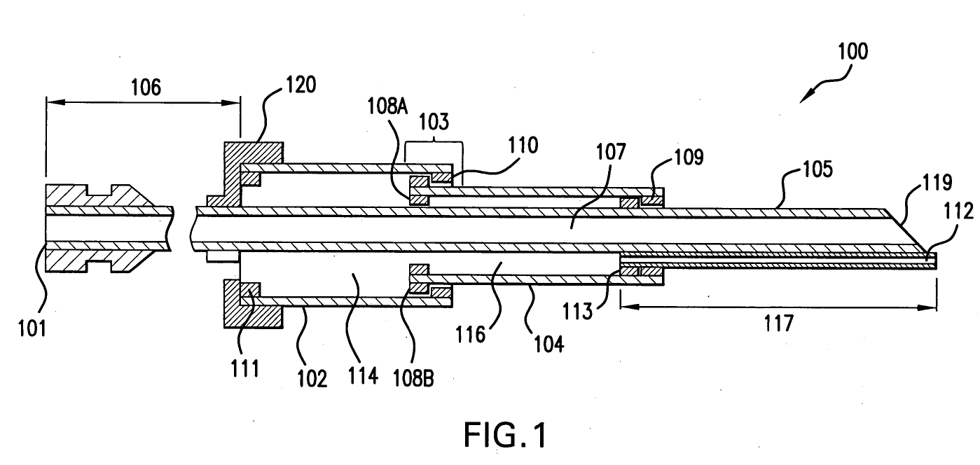

Referring now to FIG. 1, an aspiration catheter 100 is shown. Aspiration catheter 100 includes a proximal aspiration port 101, an aspiration shaft 105, a proximal tubular element 102, and a distal tubular element 104. Proximal tubular element 102 is open at both ends with a lumen 114 extending therethrough. Distal tubular element 104 is also open at both ends with a lumen 116 extending therethrough.

Aspiration shaft 105 having an aspiration lumen 107 is similar to other tubular members known in the art that are suitable for aspirating embolic or thrombotic matter from a vessel. Aspiration shaft 105 is a long, continuous tubular body having a proximal segment 106 that extends proximal of proximal tubular element 102 and a distal segment 117 that extends distal of distal tubular element 104. A cross-sectional diameter of aspiration shaft 105 is relatively large, encompassing most of the cross-sectional diameter of catheter 100. Typical diameters for aspiration shafts such as aspiration shaft 105 range from 0.7 mm to 18 mm. While the length of aspiration shaft 105 may vary depending upon the specific procedure, a typical length for aspiration shaft 105 is 145 cm.

A proximal aspiration port 101 is disposed at a proximal end of aspiration shaft 105. Proximal aspiration port 101 is adapted to be joined to a source of negative pressure, as is well-known in the art. For example, proximal aspiration port 101 may be a valve or a luer connector. The source of negative pressure may be a syringe or a line to a continuous vacuum source.

Aspiration shaft 105 may be made from any material known in the art and appropriate for use as a human-use catheter. Aspiration catheter 105 must be sufficiently strong to “telescope” the outer sheath, i.e., proximal tubular element 102 and distal tubular element 104, described in detail hereinafter, and to withstand the negative pressures associated with aspirating the vessel. Aspiration shaft 105 must also be flexible enough to navigate the tortuous pathways of the vascular system. Suitable metal materials include stainless steel and nitinol, provided that the walls of aspiration shaft 105 are thin enough to remain flexible. Suitable polymeric materials include PEBAX, polyvinyl chloride, polyethylene, polyethylene terephthalate, polyamide, or polyimide. Further, if a polymeric material is used, an optional layer of a stiffer material may be added to or embedded within the main material of aspiration shaft 105 to enhance the pushability of catheter 100. For example, a braid of metal or polymeric filaments could be included.

At a distal tip of catheter 100, aspiration shaft 105 includes a distal aspiration port 119. To increase the cross-sectional area of distal aspiration port 119 open to the vessel, in one embodiment distal port 119 is set at an oblique angle to the rest of catheter 100. Further, the distal tip of catheter 100 may include a radiopaque marker (not shown) to aid in tracking the distal tip during the procedure. Such a radiopaque marker is typically a band of radiopaque material, such as platinum, fixedly attached to the distal tip of catheter 100.

Also, in one embodiment, a short guidewire shaft 112 is disposed substantially on distal segment 117 of aspiration shaft 105. Guidewire shaft 112 is a short length of tubing of a much smaller diameter than that of aspiration shaft 105. For example, an inner diameter of guidewire shaft 112 may range from approximately 0.016 inches to approximately 0.020 inches, although this inner diameter varies according to the size of the actual guidewire intended to be used for the procedure. Guidewire shaft 112 is positioned along an outer surface of aspiration shaft 105 and is significantly shorter in length and significantly smaller in diameter than aspiration shaft 105. Guidewire shaft 112 is made of similar materials as discussed above with reference to aspiration shaft 105. Guidewire shaft 112 is open at a distal end thereof to the vessel and at a proximal end thereof to lumen 116 extending through distal tubular element 104.

Guidewire shaft 112 can be a separate tube, either polymeric or metallic, bonded or otherwise cemented to the outer surface of aspiration shaft 105. However, a distal portion of aspiration shaft 105 and guidewire shaft 112 may be formed together as a dual-lumen polymeric extrusion that is then bonded to a single lumen tube that forms a proximal portion of aspiration shaft 105. Alternatively, a dual-lumen polymeric extrusion can be used where a proximal portion of one of the lumens has been cut away such that the remaining distal portion is the guidewire lumen.

Proximal tubular element 102 is slidably mounted over aspiration shaft 105. Distal tubular element 104 is slidably mounted over aspiration shaft 105, and over guidewire shaft 112, if included, distal to proximal tubular element 102.

Proximal tubular element 102 and distal tubular element 104 are made of similar polymeric materials as aspiration shaft 105, such as polyvinyl chloride, polyethylene, polyethylene terephthalate, polyamide, or, preferably, polyimide. Proximal tubular element 102 and distal tubular element 104 can be manufactured by any method known in the art, such as by extrusion, and are preferably both made of the same material or materials.

As shown in FIG. 1, the diameter of lumen 114 extending through proximal tubular element 102 is greater than an outer diameter of distal tubular element 104. As such, distal tubular element 104 may be slidably received within proximal tubular element 102. The dimensions in FIG. 1 are exaggerated for clarity; in actual use, the inner diameter of proximal tubular element 102 and the outer diameter of distal tubular element 104 differ by a fairly small degree. The diameter of lumen 116 is sized so as to fit over aspiration shaft 105, while also providing clearance for the passage of a guidewire therethrough. Further, the walls of proximal tubular element 102 and distal tubular element 104 are relatively thin, so as to minimize the discontinuity at a joint 103 on an exterior surface of catheter 100.

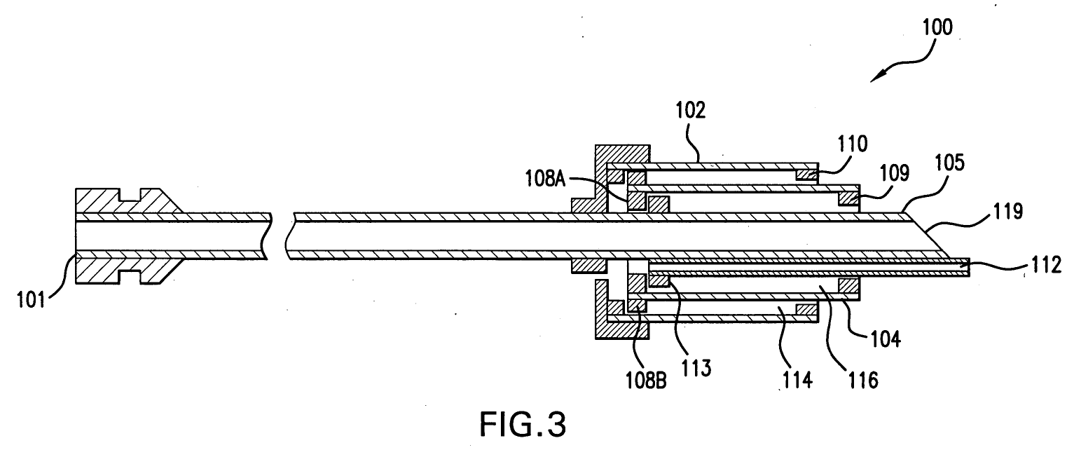

The lengths of proximal tubular element 102 and distal tubular element 104 are approximately equal. While the actual lengths thereof may vary widely, the total length of catheter 100 when fully contracted (as seen in FIG. 3) is substantially less than that of a typical guidewire. For the purposes of illustration only, a typical aspiration catheter is approximately 145 cm long. In this case, proximal tubular element 102 and distal tubular element 104 would each be approximately 50 cm in length, to compensate for the overlap between the two portions. Distal region 117 is also approximately 47 cm in length, which results in a catheter approximately 145 cm in length when fully extended.

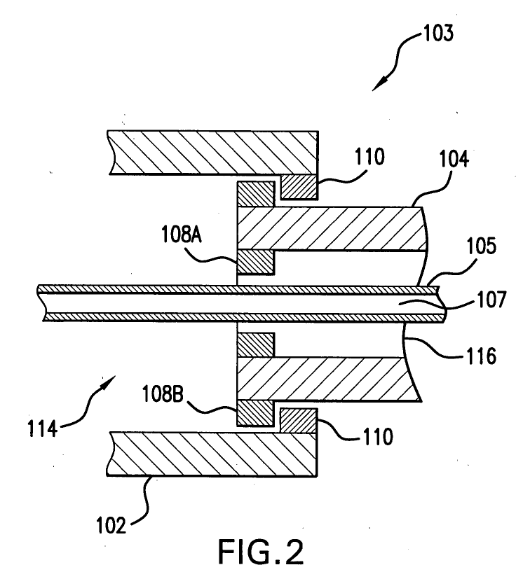

As seen more clearly in FIG. 2, the relative positions of proximal tubular element 102, distal tubular element 104, and aspiration shaft 105 are controlled using a series of stops. Proximal outer stop 108B of distal tubular element 104 is a short length of tubing, which in one embodiment is made of the same material as that of distal tubular element 104, bonded to an outer surface of distal tubular element 104. Distal stop 110 of proximal tubular element 102 is also a short length of tubing, which in one embodiment is made of the same material as that of proximal tubular element 102. Distal stop 110 is bonded to an inner surface of proximal tubular element 102 at the distal end thereof.

Proximal outer stop 108B and distal stop 110 are sized to prevent the removal of the proximal end of distal tubular element 104 from the distal end of proximal tubular element 102. In one embodiment, an inner diameter of proximal outer stop 108B is approximately equal to a diameter of lumen 114. Similarly, in one embodiment, an inner diameter of distal stop 110 is approximately equal to an outer diameter of distal tubular element 104. Therefore, proximal outer stop 108B cannot move past distal stop 110, thereby keeping the proximal end of distal tubular element 104 disposed within proximal tubular element 102.

Further, referring to FIG. 1, a proximal stop 111 is positioned to prevent the extraction of distal tubular element 104 from a proximal end of proximal tubular element 102. Proximal stop 111 is also a short length of tubing, which in one embodiment is made of the same material as that of proximal tubular element 102. Proximal stop 111 is bonded to the inner surface of proximal tubular element 102 at the proximal end thereof, and is of a similar size as distal stop 110.

Further, to prevent proximal and distal tubular elements 102, 104 from sliding off of aspiration shaft 105, an aspiration shaft stop 113 is disposed on an exterior surface of aspiration shaft 105. In one embodiment, aspiration shaft stop 113 is fixedly attached to aspiration shaft 105 at the point where guidewire shaft 112 communicates with lumen 116. In this embodiment, shown in FIG. 1, the distal end of guidewire shaft 112 is disposed within aspiration shaft stop 113. In one embodiment, aspiration stop 113 is made from the same material at aspiration shaft 105, and an outer diameter of aspiration stop 113 is approximately equal to the diameter of distal tubular element lumen 116.

A distal tubular element proximal inner stop 108A is a short length of tubing similar to distal tubular element proximal outer stop 108B bonded to an inner surface of distal tubular element 104 at a proximal end thereof. Distal tubular element proximal inner stop 108A is sized to prevent aspiration stop 113 from being extracted from distal tubular element 104 as well as to have a close but sliding fit with aspiration shaft 105 to minimize backbleeding. Similarly, a distal tubular element distal stop 109 is a short length of tubing made of a similar material as that of distal tubular element proximal inner stop 108A bonded to the inner surface of distal tubular element 104 at a distal end thereof. Distal tubular element distal stop 109 is sized to prevent aspiration stop 113 from being extracted from distal tubular element 104 as well as to have a close but sliding fit with aspiration shaft 105. In one embodiment, distal tubular element distal stop 109 is approximately equal in diameter to an outer diameter of aspiration shaft 105. However, a guidewire must pass between distal tubular element proximal stop 108A and aspiration shaft 105. As such, the diameter of distal tubular element proximal inner stop 108A must be less than that of the outer diameter of aspiration shaft 105, so that sufficient clearance for a guidewire to pass therebetween is maintained. Alternatively, distal tubular element proximal inner stop 108A may contain a hole or series of holes therein through which a guidewire may be threaded (not shown).

Although all stops described with respect to FIG. 1 are shown at the proximal or distal ends of proximal tubular element 102 and distal tubular element 104, the placement of the stops need not be so arranged. In order to control the length of catheter 100 in either the fully extended position (shown in FIG. 1) or in the nested position (shown in FIG. 3), the stops may be placed anywhere along the lengths of tubular elements 102, 104; however, the placement of the stops on the ends thereof achieves a maximum length for catheter 100. Further, the function of the stops described herein is to prevent the complete extraction of distal tubular element 104 from proximal tubular element 102. However, other structures may be used for this purpose, such as increasing the outer diameter of distal tubular element 104 at the proximal and distal ends thereof, and/or coating the inner surface of proximal tubular element 102 and/or the outer surface of distal tubular element 104 at the proximal and distal ends thereof with a rough material.

Additionally, in order to prevent backbleeding in the space between aspiration shaft 105 and each of the telescoping portions, proximal tubular element 102 and distal tubular element 104, and in the space between the telescoping portions themselves, all stops discussed herein are sized to have a close but sliding fit with the tubular elements against which the stops slide. To further limit backbleeding, and to provide a gripping surface, an optional flanged hub 120 may be included on proximal tubular element 102. Flanged hub 120 is made from any body-compatible material, such as stainless steel or a suitable polymer, such as polyimide. Flanged hub 120 is fixedly attached to a proximal end of proximal tubular element 102, such as by cementing. A proximal end of flanged hub 120 has a close but sliding fit with aspiration shaft 105.

Catheter 100 is used in the following manner. For the purposes of example only, a specific procedure using a distal protection filter is described. However, catheter 100 may be used in a similar manner in any procedure where an aspiration catheter is inserted over a guidewire into a patient.

In a stent-delivery procedure, a guidewire is inserted into a patient's vascular system and steered to a treatment site in a vessel. The guidewire includes a distal protection filter or occluder, which is positioned downstream of the treatment site to capture any embolic particles dislodged during stent delivery. At some point during the procedure, the build-up of embolic particles in the vessel due to the distal protection element (either a filter or an occluder) may become onerous, such as by blocking a filter and occluding a vessel, and the embolic particles must then be removed.

Catheter 100 is provided in the nested position shown in FIG. 3. A proximal end of the guidewire is threaded into the open distal end of guidewire shaft 112 and passed therethrough into lumen 116. Finally, the proximal end of the guidewire is threaded past distal tube inner proximal stop 108A alongside aspiration shaft 105, into lumen 114, and out a proximal end of proximal tubular element 102.

While holding the proximal end of the guidewire, a clinician grasps aspiration shaft 105 along some portion thereof protruding from the nesting proximal and distal tubes 102, 104. For example, the clinician may grasp proximal aspiration port 101. Further, to prevent proximal tubular element 102 from being carried into the vessel, a proximal end thereof should also be grasped. While holding the guidewire and proximal tubular element 102 steady, the clinician pushes aspiration shaft 105 distally. As aspiration shaft 105 moves into the vascular system, aspiration shaft stop 113 abuts distal tubular element distal stop 109 once distal segment 117 of aspiration shaft 105 has been extended from distal tubular element 104. As aspiration shaft 105 is pushed further into the vascular system, distal tubular element 104 is moved distally, telescoping distal tubular element 104 outward from proximal tubular element 102. Finally, aspiration catheter 100 attains the fully expanded configuration shown in FIG. 1, and distal aspiration port 119 is positioned just proximal of or within the distal protection filter. While catheter 100 is being telescoped into the vessel, the guidewire is maintained within guidewire shaft 112 and lumens 114 and 116 to guide catheter 100 to the treatment site.

A negative pressure source such as a syringe is attached to proximal aspiration port 101. Negative pressure is applied to proximal aspiration port 101, and the embolic material captured within the distal protection filter is drawn through distal aspiration port 119, into aspiration lumen 107, and out of proximal aspiration port 101 for disposal.

After aspiration is complete, aspiration catheter 100 is removed from the patient so that other therapeutic or diagnostic catheters may be introduced to the treatment site over the guidewire. To extract aspiration catheter 100 quickly and easily, aspiration catheter 100 is returned to the nested configuration, shown in FIG. 3. The clinician grasps proximal aspiration port 101 and draws aspiration shaft 105 proximally, thereby pulling aspiration shaft distal segment 117 into distal tubular element 104 and distal tubular element 104 into proximal tubular element 102 in a telescoping manner. Aspiration shaft 105 is prevented from being pulled entirely through distal tubular element 104 by the abutment of aspiration shaft stop 113 with distal tubular element proximal inner stop 108A. Similarly, distal tubular element 104 is prevented from being pulled through the open proximal end of proximal tubular element 102 by the abutment of distal tubular element proximal outer stop 108B with proximal tubular element proximal stop 111. After the nesting of aspiration catheter 100 is complete, the effective over-the-wire length of catheter 100 is such that the clinician may withdraw catheter 100 without losing contact with the proximal end of the guidewire.

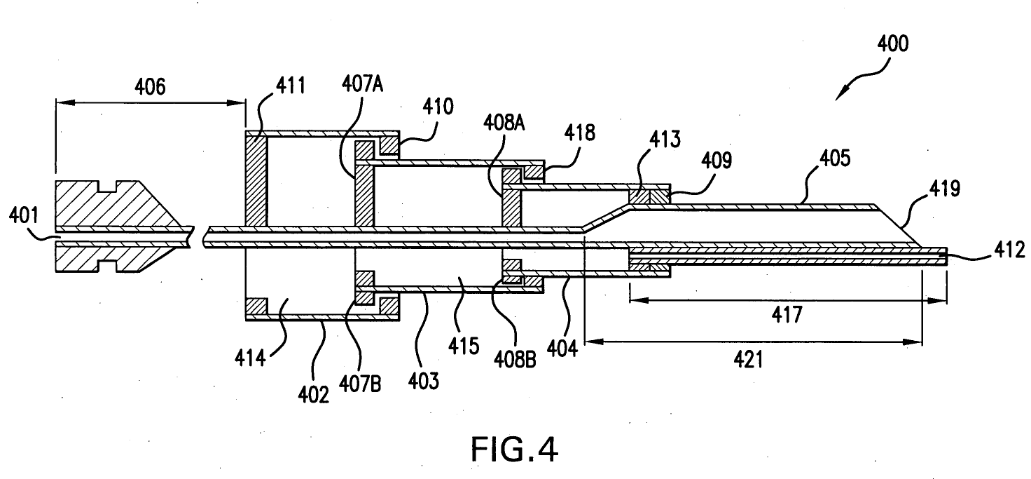

Referring now to FIG. 4, an alternate embodiment of an aspiration catheter 400 according to the present invention is shown. Catheter 400 includes a proximal aspiration port 401, a proximal tubular element 402 defining a lumen 414, a middle tubular element 403 defining a lumen 415, and a distal tubular element 404 defining a lumen 416. In this embodiment, an effective over-the-wire length of aspiration catheter 400 can be reduced to be significantly less than that of the dual-element design of aspiration catheter 100. However, an outer diameter of proximal tubular element 402 will be larger than that of proximal tubular element 102 (described above) due to the requisite nesting of both middle tubular element 403 and distal tubular element 404 within proximal tubular element 402, if an inner lumen of distal tubular element 404 is the same as that of distal tubular element 104.

Aspiration catheter 400 is similar in construction with aspiration catheter 100. Aspiration shaft 405 is a long tube made from similar materials as aspiration shaft 105, described above. However, in this embodiment, a distal portion 421 of aspiration shaft 405 has a larger diameter than a proximal portion, or the remainder, of aspiration shaft 405. A larger diameter near distal aspiration port 419 is desirable, so that a large volume may be aspirated. The larger diameter does not extend the entire length of aspiration shaft 405, and the smaller diameter in the proximal portion improves the flexibility thereof. Additionally, distal portion 421 may be made of a very stiff material, such as a metal, to increase the pushability of aspiration shaft 405.

The same or similar materials used to form tubular elements 102, 104 are used to form tubular elements 402, 403, 404. In one embodiment, the material is polyimide. Further, as with catheter 100, in one embodiment, the material used for catheter 400 includes a reinforcing layer, such as a metal braid, embedded within the main polymer.

As with catheter 100, the relative positions of proximal tubular element 402, middle tubular element 403, and distal tubular element 404 are controlled using a series of stops. In one embodiment, all stops are short lengths of tubing made of the same or similar material as that of the tubular elements 402, 403, 404 to which they are bonded, although the stop may be of any structure known in the art. The bonding can be of any manner known in the art, such as cementing or heat treatment.

As seen in FIG. 4, a distal tubular element proximal inner stop 408A is bonded to an inner surface of distal tubular element 404 on a proximal end thereof. A distal tubular element proximal outer stop 408B is bonded to an outer surface of distal tubular element 404 on the proximal end thereof. A distal tubular element distal stop 409 is bonded to an inner surface of distal tubular element 404 on a distal end thereof.

A middle tubular element distal stop 418 is bonded to the inner surface of middle tubular element 403 on a distal end thereof. A middle tubular element proximal inner stop 407A is bonded to an inner surface of middle tubular element 403 on a proximal end thereof. A middle tubular element proximal outer stop 407B is bonded to an outer surface of middle tubular element 403 at the proximal end thereof.

A proximal tubular element distal stop 410 is bonded to an inner surface of proximal tubular element 402 at a distal end thereof. Finally, a proximal tubular element proximal stop 411 is bonded to an inner surface of proximal tubular element 402 at a proximal end thereof.

Middle tubular element proximal outer stop 407B and proximal tubular element distal stop 410 are sized to prevent the removal of the proximal end of middle tubular element 403 from the distal end of proximal tubular element 402. In one embodiment, an outer diameter of middle tubular element proximal outer stop 407B is approximately equal to a diameter of lumen 414. Similarly, in one embodiment, an inner diameter of proximal tubular element distal stop 410 is approximately equal to an outer diameter of middle tubular element 403.

Middle tubular element proximal outer stop 407B and proximal tubular element proximal stop 411 are sized to prevent the removal of the proximal end of middle tubular element 403 from the proximal end of proximal tubular element 402. Thus, in one embodiment, an inner diameter of proximal tubular element proximal stop 411 is approximately equal to the outer diameter of middle tubular element 403.

Distal tubular element proximal outer stop 408B and middle tubular element distal stop 418 are sized to prevent the removal of the proximal end of distal tubular element 404 from the distal end of middle tubular element 403. In one embodiment, an outer diameter of distal tubular element proximal outer stop 408B is approximately equal to that of lumen 415. Similarly, in one embodiment, an inner diameter of middle tubular element distal stop 418 is approximately equal to an outer diameter of distal tubular element 404.

Middle tubular element proximal inner stop 407A and distal tubular element proximal outer stop 408B are sized to prevent the removal of the proximal end of distal tubular element 404 from the proximal end of middle tubular element 403.

Distal tubular element proximal inner stop 408A and aspiration shaft stop 413 are sized to restrict the longitudinal movement of aspiration shaft 405 within distal tubular element 404. In other words, distal tubular element proximal inner stop 408A and aspiration stop 413 prevent aspiration shaft 405 from being withdrawn proximally from distal tubular element 404. In one embodiment an outer diameter of aspiration stop 413 is approximately equal that of lumen 416. However, distal tubular element proximal inner stop 408A must be sized so as to allow a guidewire to pass between it and aspiration shaft 405. Alternatively, distal tubular element proximal inner stop 408A may contain a hole or series of holes therein through which a guidewire may be threaded (not shown).

Catheter 400 is shown in a fully extended position in FIG. 4, when middle tubular element proximal outer stop 407B and proximal tubular element distal stop 410 abut one another, distal tube proximal outer stop 408B and middle tubular element distal stop 418 abut each other, and distal tubular element distal stop 409 and aspiration stop 413 abut one another.

Catheter 400 is in a first partially extended position (not shown) when middle tubular element proximal outer stop 407B and proximal tubular element distal stop 410 abut one another, distal tube proximal outer stop 408B and middle tubular element distal stop 418 abut each other, but distal tubular element distal stop 409 and aspiration stop 413 do not abut one another.

Catheter 400 is in a second partially extended position (not shown) when middle tubular element proximal outer stop 407B and proximal tubular element distal stop 410 abut one another and distal tubular element distal stop 409 and aspiration stop 413 abut one another, but distal tube proximal outer stop 408B and middle tubular element distal stop 418 do not abut each other.

Catheter 400 is in a third partially extended position (not shown) when distal tube proximal outer stop 408B and middle tubular element distal stop 418 abut each other and distal tubular element distal stop 409 and aspiration stop 413 abut one another, but middle tubular element proximal outer stop 407B and proximal tubular element distal stop do not abut one another.

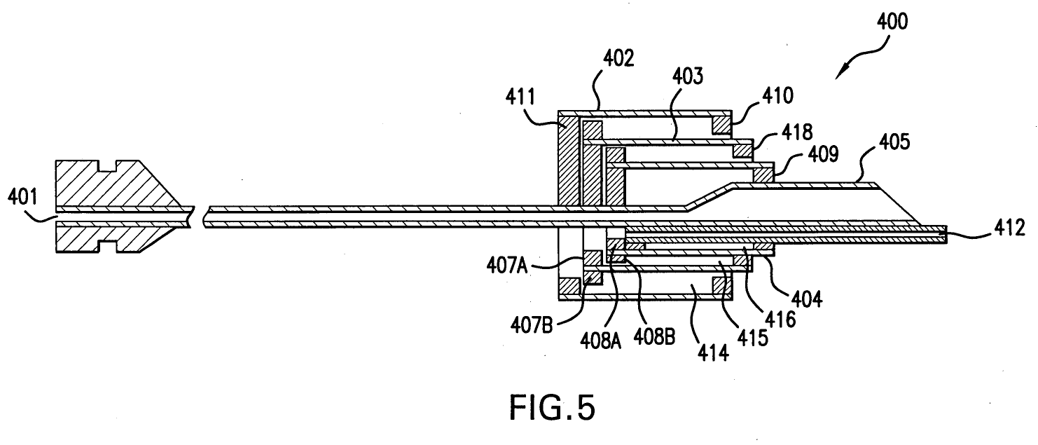

Catheter 400 is in a fully nested position, shown in FIG. 5, when proximal tubular element proximal stop 411 abuts middle tubular element outer stop 407B, middle tubular element proximal inner stop 407A abuts distal tubular element proximal outer stop 408B, and distal tubular element proximal inner stop 408A abuts aspiration stop 413.

The operation and use of aspiration catheter 400 is very similar to that of catheter 100, described above. When determining the effective over-the-wire length of catheter 100 after insertion, the clinician can choose to extend catheter 400 to any of the lengths available: fully extended or partially extended. Also, for rapid exchange, the clinician may choose to fully retract catheter 400 by drawing aspiration shaft 405 proximally until catheter 400 is in the fully nested position, or only partially, until one of the partially extended positions is achieved.

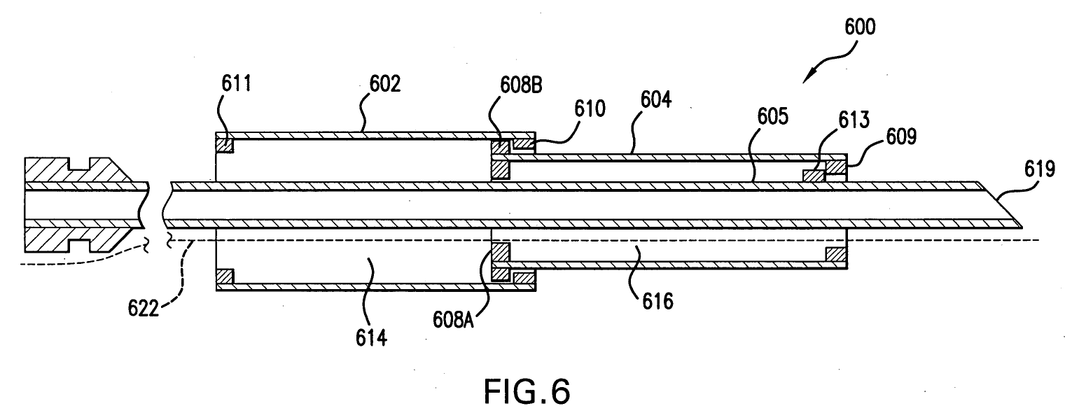

In another embodiment, shown in FIG. 6 and similar to that shown in FIG. 1, guidewire shaft 112 is eliminated from the design of a catheter 600. In this embodiment, a proximal tubular element 602 and a distal tubular element 604, which are slidingly disposed over an aspiration shaft 605, are slightly longer than in the embodiment described with respect to FIG. 1, as no significant length of aspiration shaft 605 projects distally from distal tubular element 604 when catheter 600 is in a fully extended position, as shown in FIG. 6. Catheter 600 may also include a middle tubular element, such as is described above with respect to FIG. 4.

In this embodiment, an aspiration stop 613 is disposed on an aspiration shaft 605 close to a distal aspiration port 619 so that catheter 600 may be advanced over a guidewire. In one embodiment, aspiration shaft stop 613 is sized so as to provide clearance between aspiration stop 613 and an inner wall of a distal tubular element 604. As shown in FIG. 6, this is achieved by using a half-section of tubing for aspiration stop 613, so that a distal tubular element lumen 616 is only blocked on one side of aspiration shaft 605. Further, a distal tubular element distal stop 609 and a distal tubular element proximal inner stop 608A are sized to allow for sufficient clearance for a guidewire to pass between stops 609, 608A and aspiration shaft 605. As such, a clear path for a guidewire exists from a distal end of distal tubular element 604, through lumen 616, therethrough to a proximal tubular element lumen 614, and out a proximal end of proximal tubular element 602. A guidewire 622 is shown in phantom to clearly demonstrate the guidewire path. In another embodiment, not shown, aspiration shaft stop 613 includes a hole or a series of holes through which a guidewire may be threaded. In all other respects, catheter 600 is the same in structure and use as either catheter 100 or catheter 400, described above.

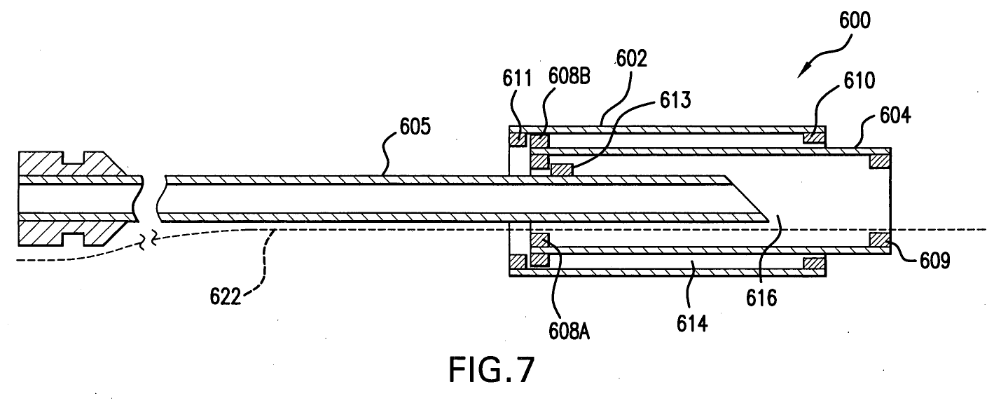

Alternatively, aspiration catheter 600 may be used without a guidewire. As such, a clinician inserts a distal end of aspiration catheter 600 into a patient's vascular system. Aspiration catheter 600 is in the collapsed or nested configuration shown in FIG. 7. A proximal end of proximal tubular element 602 is grasped by the clinician and aspiration shaft 605 is pushed distally so that aspiration shaft stop 613 abuts distal tubular element distal stop 609. As distal tubular element 604 is pushed distally, i.e., telescoped from within lumen 614, aspiration catheter 600 is simultaneously steered through the vascular system. Aspiration catheter 600 may or may not be fully telescoped. Aspiration shaft 605 continues to be pushed distally until distal aspiration port 619 reaches the desired treatment location.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. All patents and publications discussed herein are incorporated in their entirety by reference thereto.

1. An aspiration catheter comprising:
an aspiration shaft;
a proximal tube having a first lumen extending therethrough, wherein said proximal tube is slidably disposed over said aspiration shaft; and
a distal tube having a second lumen extending therethrough, wherein said distal tube is slidably disposed over said aspiration shaft distal to said proximal tube, said distal tube is slidably positionable within said first lumen, at least a proximal end of said distal tube is unremovably disposed within said first lumen, and said aspiration shaft is unremovable from said second lumen.

2. The aspiration catheter according to claim 1, wherein a distal aspiration port disposed at a distal tip of said aspiration shaft is set at an oblique angle.

3. The aspiration catheter according to claim 1, wherein a diameter of a distal portion of said aspiration shaft is larger than a diameter of a proximal portion of said aspiration shaft.

4. The aspiration catheter according to claim 1, further comprising a guidewire shaft having a guidewire lumen disposed adjacent to said aspiration shaft.

5. The aspiration catheter according to claim 4, wherein said guidewire shaft is disposed along a distal segment of said aspiration shaft.

6. The aspiration catheter according to claim 1, further comprising:
a first proximal stop fixedly attached to an interior surface of said proximal tube on a proximal end thereof;
a first distal stop fixedly attached to the interior surface of said proximal tube on a distal end thereof;
a second proximal stop fixedly attached to an exterior surface of said distal tube on a proximal end thereof;
a third proximal stop fixedly attached to an interior surface of said distal tube on a proximal end thereof;
a second distal stop fixedly attached to the interior surface of said distal tube on a distal end thereof; and
a fourth proximal stop fixedly attached to an exterior surface of said aspiration shaft in a distal region thereof.

7. An aspiration catheter comprising:
an aspiration shaft;
a proximal tube having a first lumen extending therethrough, wherein said proximal tube is slidably disposed over said aspiration shaft;
a middle tube having a second lumen extending therethrough, wherein said middle tube is slidably disposed over said aspiration shaft distal to said proximal tube, said middle tube is slidably positionable within said first lumen, and at least a proximal end of said middle tube is unremovably disposed within said first lumen; and
a distal tube having a third lumen extending therethrough, wherein said distal tube is slidably disposed over said aspiration shaft distal to said middle tube, said distal tube is slidably positionable within said second lumen, at least a proximal end of said distal tube is unremovably disposed within said second lumen, and said aspiration shaft is unremovable from said third lumen.

8. The aspiration catheter according to claim 7, further comprising a guidewire shaft having a guidewire lumen disposed adjacent to said aspiration shaft.

9. The aspiration catheter according to claim 7, further comprising:
a first proximal stop fixedly attached to an interior surface of said proximal tube on a proximal end thereof;
a first distal stop fixedly attached to the interior surface of said proximal tube on a distal end thereof;
a second proximal stop fixedly attached to an exterior surface of said middle tube on a proximal end thereof;
a third proximal stop fixedly attached to an interior surface of said middle tube on the proximal end thereof;
a second distal stop fixedly attached to the interior surface of said middle tube on a distal end thereof;
a fourth proximal stop fixedly attached to an exterior surface of said distal tube on a proximal end thereof;
a fifth proximal stop fixedly attached to an interior surface of said distal tube on the proximal end thereof;
a third distal stop fixedly attached to the interior surface of said distal tube of a distal end thereof; and
a sixth proximal stop fixedly attached to an exterior surface of said aspiration shaft in a distal region thereof.

10. A method of aspirating a vessel comprising:
introducing a guidewire into the vessel;
providing an aspiration catheter having an aspiration shaft slidably connected to a telescoping outer sheath, wherein said telescoping outer sheath is in a fully contracted configuration;
inserting a proximal end of the guidewire into the telescoping outer sheath;
pushing said aspiration shaft distally, thereby causing said telescoping outer sheath to expand;
maneuvering said aspiration catheter along said guidewire to a treatment site; and
aspirating the vessel.

11. The method of aspirating a vessel according to claim 10, wherein said aspiration catheter includes a guidewire shaft having a guidewire lumen, said guidewire shaft being disposed on a distal segment of said aspiration shaft, and said guidewire is threaded through said guidewire lumen into said telescoping sheath.

12. The method of aspirating a vessel according to claim 10, further comprising:
pulling said aspiration shaft proximally, thereby causing said telescoping outer sheath to contract; and
removing said aspiration catheter from the guidewire.

13. A method of aspirating a vessel comprising:
providing an aspiration catheter having an aspiration shaft slidably connected to a telescoping outer sheath, wherein said telescoping outer sheath is in a fully contracted configuration;
inserting a distal end of said aspiration catheter into said vessel;
pushing said aspiration shaft distally, thereby causing said telescoping outer sheath to expand;
maneuvering said aspiration catheter through said vessel to a treatment site; and
aspirating the vessel.

14. The method of aspirating a vessel according to claim 13, further comprising:
pulling said aspiration shaft proximally, thereby causing said telescoping outer sheath to contract; and
removing said aspiration catheter from said vessel.
